(19) United States
(12) Patent Application Publication
(10) Pub. No.: US 2025/0127986 A1
(43) Pub. Date: Apr. 24, 2025

(54) INFUSION PUMP SYSTEM AND METHOD WITH FLUID LINE AUTO FLUSH

(71) Applicant: ICU MEDICAL, INC., San Clemente, CA (US)

(72) Inventors: Jeffrey James Gylland, San Clemente, CA (US); Gerald William Brann, San Clemente, CA (US); James Duane Jacobson, San Clemente, CA (US); Arthur E. Webb, San Clemente, CA (US); James Cudney, San Clemente, CA (US); Nursel Asikhan-Berlinguette, San Clemente, CA (US)

(21) Appl. No.: 18/988,342

(22) Filed: Dec. 19, 2024

Related U.S. Application Data
(63) Continuation of application No. 17/671,227, filed on Feb. 14, 2022, now Pat. No. 12,201,811.
Application No. 17/671,227 is a continuation of application No. 16/301,379, filed on Nov. 13, 2018, now Pat. No. 11,246,985.
(60) Provisional application No. 62/336,191, filed on May 13, 2016.

(51) Int. Cl. A61M 5/172; A61M 5/14; A61M 5/142; A61M 5/145; A61M 5/168; G05B 19/042; G05D 7/06; G06F 3/0481; G16H 20/17; G16H 40/67; G16H 50/20

(52) U.S. Cl. CPC A61M 5/172; A61M 5/14; A61M 5/1407; A61M 5/142; A61M 5/145; A61M 5/168; A61M 5/16827; A61M 5/16831; G05B 19/042; G05D 7/0635; G16H 20/17; G16H 40/67; G16H 50/20; A61M 2005/1403; A61M 2205/3331; A61M 2205/3334; A61M 2205/3379; A61M 2205/50; A61M 2205/502; A61M 2205/52; G05B 2219/25312; G06F 3/0481

(57) ABSTRACT

An infusion pump system and method with common line auto flush, wherein the infusion pump system has a reservoir, a fluid line in fluid connection with the reservoir and having a terminal fluid delivery end, and an infusion pump. The method includes receiving an instruction to flush the fluid line at a flush rate, wherein the fluid line comprises a fluid line volume of a second fluid; receiving an instruction to infuse the first fluid at an infusion rate; flushing the fluid line by infusing a fluid line volume of the first fluid from the reservoir at a flush rate greater than the infusion rate; determining that the fluid line volume of the first fluid has been infused at the flush rate; and changing to infusing the first fluid through from the reservoir at the infusion rate.

### CROSS-REFERENCE TO RELATED APPLICATIONS

The present application is a continuation of U.S. application Ser. No. 17/671,227, filed Feb. 14, 2022, which is a continuation of U.S. application Ser. No. 16/301,379, filed Nov. 13, 2018, which is the national stage of International Application No. PCT/US2017/032017, filed May 10, 2017, which claims the benefit of priority from U.S. Provisional No. 62/336,191, filed May 13, 2016, all of which are incorporated by reference in their entireties.

### BACKGROUND OF THE INVENTION

### Field of the Invention

The present invention relates to medical devices. More specifically, the invention relates to infusion pump systems.

Infusion pumps are medical devices that deliver fluids, including nutrients and medications such as antibiotics, chemotherapy drugs, and pain relievers, in controlled amounts. Many types of pumps, including large volume, patient-controlled analgesia (PCA), elastomeric, syringe, enteral, and insulin pumps, are used worldwide in healthcare facilities, such as hospitals, and in the home. Clinicians and patients rely on pumps for safe and accurate administration of fluids and medications.

It is desirable to provide more than one therapeutic fluid from the infusion pump for some treatment regimens. Presently, two fluid reservoirs with different therapeutic fluids are connected to the infusion pump and then delivered through a common line having a terminal fluid delivery end. The first therapeutic fluid and second therapeutic fluid are administered alternately by switching the fluid flow path between the first reservoir and the second reservoir.

Unfortunately, the therapeutic fluid remaining in the common line creates problems when switching between the two therapeutic fluids. First, the remaining therapeutic fluid must be cleared from the common line before the next therapeutic fluid begins administration, delaying the next therapeutic fluid from reaching the patient. Second, when the therapeutic fluids are administered at different rates, the therapeutic fluid remaining in the common line will be administered at the rate of the new fluid being administered, e.g., the remaining first therapeutic fluid will be administered at the rate specified for the second therapeutic fluid. This can result in the patient from receiving more or less than the optimum therapy with respect to the first therapeutic fluid. Third, the remaining therapeutic fluid may not be correctly accounted for, potentially creating errors in the values indicated at the infusion pump. While the pump data will be correct in terms of infusion rates over given times, the actual fluid delivery to the terminal fluid delivery end at the patient is not correctly captured in pump and system data.

It would be desirable to have infusion pump systems and methods with common line auto flush that would overcome the above disadvantages.

### SUMMARY OF THE DISCLOSURE

One aspect of the present invention provides a method to infuse fluids with an infusion pump system, the infusion pump system having a first reservoir containing a first fluid, a second reservoir containing a second fluid, a junction in fluid communication with the first reservoir and the second reservoir, a common line in fluid communication with the junction at one end and having a terminal fluid delivery end, and an infusion pump operable to drive fluid through the common line. The method includes infusing the first fluid at a first rate along a first flow path including the first reservoir, the junction, and the common line; entering a common line flush volume value for the common line; switching from the first flow path to a second flow path including the second reservoir, the junction, and the common line; driving the second fluid at the first rate along the second flow path; monitoring volume of the second fluid driven at the first rate; and driving the second fluid at a second rate along the second flow path when the monitored volume is equal to or greater than the common line flush volume value.

Another aspect of the present invention provides an infusion pump system including a first reservoir containing a first fluid; a second reservoir containing a second fluid; a junction in fluid communication with the first reservoir and the second reservoir; a common line in fluid communication with the junction at one end and a terminal fluid delivery end; and an infusion pump operable to drive fluid through the common line. The infusion pump is operable to infuse the first fluid at a first rate along a first flow path including the first reservoir, the junction, and the common line; receive a common line flush volume value for the common line; switch from the first flow path to a second flow path including the second reservoir, the junction, and the common line; drive the second fluid at the first rate along the second flow path; monitor volume of the second fluid driven at the first rate; and drive the second fluid at a second rate along the second flow path when the monitored volume is equal to or greater than the common line flush volume value.

Yet another aspect of the present invention provides an infusion pump, the infusion pump being operably connected to a common line in fluid communication with a junction at one end and having a terminal fluid delivery end, the junction being in fluid communication with a first reservoir containing a first fluid and a second reservoir containing a second fluid. The infusion pump includes a memory operable to store programming code; a flow controller operably connected to the memory; and a fluid driver operably connected to receive a control signal from the flow controller, the fluid driver being operable to drive fluid through the common line. The flow controller is operable to execute the programming code and provide the control signal to the fluid driver in response to the programming code. The fluid driver is responsive to the control signal to infuse the first fluid at a first rate along a first flow path including the first reservoir, the junction, and the common line; receive a common line flush volume value for the common line; switch from the first flow path to a second flow path including the second reservoir, the junction, and the common line; drive the second fluid at the first rate along the second flow path; monitor volume of the second fluid driven at the first rate; and drive the second fluid at a second rate along the second flow path when the monitored volume is equal to or greater than the common line flush volume value.

The foregoing and other features and advantages of the invention will become further apparent from the following detailed description of the presently preferred embodiments, read in conjunction with the accompanying drawings. The detailed description and drawings are merely illustrative of the invention rather than limiting. The scope of the invention is defined by the appended claims and equivalents thereof.

In certain embodiments, a control system can control operation of an infusion pump system. The infusion pump system can include a first reservoir that can hold a first fluid, a second reservoir configured to hold a second fluid, a junction in fluid communication with the first reservoir and the second reservoir, a common line in fluid communication with the junction and having a terminal fluid delivery end, and an infusion pump operable to drive fluid through the common line toward the terminal fluid delivery end. The control system can include one or more hardware processors for executing instructions. The control system can receive instructions for delivery of a first fluid at a first rate followed by a second fluid at a second rate. The control system can further infuse a first fluid at a first rate along a first flow path. The control system can also determine a first volume to clear the first fluid from a common line. The control system can infuse a second fluid at the first rate along a second flow path. The control system can monitor a second volume of the second fluid infused at the first rate. The control system can determine when the monitored volume of the second fluid meets or exceeds the first volume. The control system can change infusion of the second fluid to a second rate along the second flow path based on the determination when the monitored volume of the second fluid meets or exceeds the first volume.

The control system of the preceding paragraph can have any sub-combination of the following features: wherein the first volume is received from a user input; wherein the first volume is stored in a memory; wherein the first volume is retrieved over a network; wherein the first volume is predetermined; wherein the first volume is based on the first fluid; wherein the first rate is different than the second rate; wherein the instructions for the delivery are received from an input via a user interface; wherein the one or more hardware processors can automatically generate a user interface configured to receive an input for the first volume based on a determination of sequential delivery of two different fluids; wherein the one or more hardware processors can control a valve, wherein the valve is configured to switch the infusion of the first fluid along the first flow path to the infusion of the second fluid along the second path; wherein the one or more hardware processors are configured to transmit a control signal to begin the infusion; wherein the one or more hardware processors are configured to transmit a control signal to stop the infusion.

A method for controlling operation of an infusion pump system, the infusion pump system comprising a first reservoir configured to hold a first fluid, a second reservoir configured to hold a second fluid, a junction in fluid communication with the first reservoir and the second reservoir, a common line in fluid communication with the junction and having a terminal fluid delivery end, and an infusion pump operable to drive fluid through the common line toward the terminal fluid delivery end. The method can include receiving instructions for delivery of a first fluid at a first rate followed by a second fluid at a second rate. The method can also include infusing a first fluid at a first rate along a first flow path. The method can further include determining a first volume to clear the first fluid from a common line. In some embodiments, the method includes infusing a second fluid at the first rate along a second flow path. The method can include monitoring a second volume of the second fluid infused at the first rate. The method can also include determining when the monitored volume of the second fluid meets or exceeds the first volume. The method also includes changing infusion of the second fluid to a second rate along the second flow path based on the determination when the monitored volume of the second fluid meets or exceeds the first volume.

The method of preceding paragraph can have any sub-combination of the following features: wherein the first volume is received from a user input; wherein the first volume is stored in a memory; wherein the first volume is retrieved over a network; wherein the first volume is predetermined; wherein the first volume is based on the first fluid; wherein the first rate is different than the second rate; wherein the instructions for the delivery are received from an input via a user interface; further comprising automatically generating a user interface configured to receive an input for the first volume based on a determination of sequential delivery of two different fluids; further comprising controlling a valve, wherein the valve is configured to switch the infusion of the first fluid along the first flow path to the infusion of the second fluid along the second path; further comprising transmitting a control signal to begin the infusion; further comprising transmitting a control signal to stop the infusion.

### BRIEF DESCRIPTION OF THE DRAWINGS

FIGS. 1A & 1B are block diagrams of infusion pump systems with common line auto flush in accordance with the present invention.

FIG. 2 is a block diagram of an infusion pump with common line auto flush in accordance with the present invention.

FIG. 3 is a schematic diagram of an infusion pump with common line auto flush in accordance with the present invention.

FIGS. 4A-4O are schematic diagrams of a screen for an infusion pump system with common line auto flush in accordance with the present invention.

FIGS. 5A & 5B are graphs of fluid volume delivered at the terminal fluid delivery end of the common line versus time for a method of use for an infusion pump with common line auto flush in accordance with the present invention.

FIG. 6 is a flowchart of a method of use for an infusion pump system with common line auto flush in accordance with the present invention.

FIGS. 7A-7E are schematic diagrams of use for an infusion pump system with common line auto flush in accordance with the present invention.

Like elements share like reference numbers throughout the various figures.

### DETAILED DESCRIPTION OF THE PREFERRED EMBODIMENT

Systems and methods that improve an infusion pump system with common line are described herein. The infusion pump can deliver a first fluid from a first reservoir, then switch to delivering a second fluid from a second reservoir as per patient requirements. As discussed above, switching may result in some of the first fluid remaining in a common line. Furthermore, delivering fluids at rates other than the desired rates may result in air in the line or inaccurate therapy, which can be fatal to the patients. The systems and methods described here can improve delivery and accurately account for the first fluid remaining in the internal volume of the common line. Fluid as used herein can be any fluid suitable to be administered to a patient by infusion, including saline fluid, fluid including a drug or other therapeutic agent, or the like.

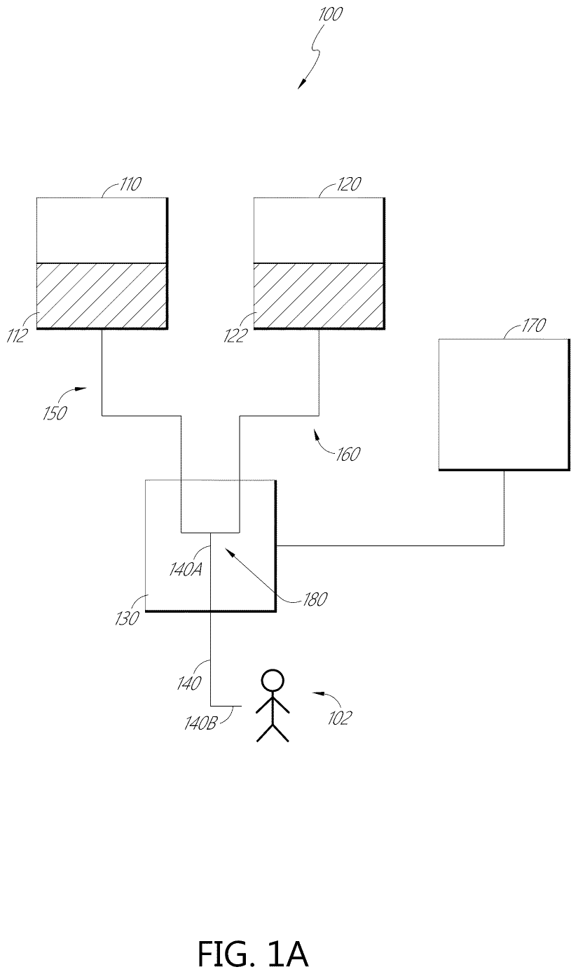

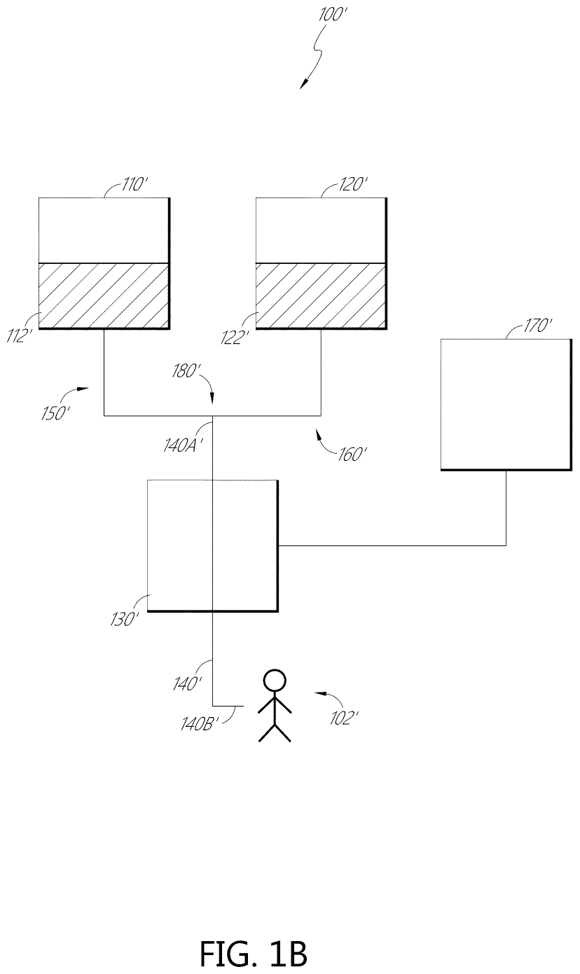

FIGS. 1A & 1B are block diagrams for embodiments of infusion pump systems with common line. The infusion pump system illustrated in FIG. 1A includes a junction in fluid communication with the first reservoir and the second reservoir internal to the infusion pump, while the embodiment of the infusion pump system illustrated in FIG. 1B has a junction in fluid communication with the first reservoir and the second reservoir external to the infusion pump. The location of the junction, in part, determines the length and internal volume of the common line between the junction and the terminal fluid delivery end. The internal shape, which is usually substantially circular, and the diameter of the common line are factors along with the length that determine the internal volume, although other shapes can be used without detracting from the scope of the disclosure.

The infusion pump system 100 of FIG. 1A includes a junction 180 internal to the infusion pump 130. The infusion pump system 100 includes a first reservoir 110 that can contain a first fluid 112; a second reservoir 120 that contain a second fluid 122; a junction 180 in fluid communication with the first reservoir 110 and the second reservoir 120; a common line 140 in fluid communication with the junction 180 at one end 140A and having a terminal fluid delivery end 140B for connection to the patient 102, and an infusion pump 130 operable to drive fluid through the common line 140. The infusion pump 130 is operable to: infuse the first fluid 112 at a first rate along a first flow path 150 including the first reservoir 110, the junction 180, and the common line 140; receive a common line flush volume value for the common line 140; switch from the first flow path 150 to a second flow path 160 including the second reservoir 120, the junction 180, and the common line 140; drive the second fluid 122 at the first rate along the second flow path 160; monitor volume of the second fluid 122 driven at the first rate; and drive the second fluid 122 at a second rate along the second flow path 160 when the monitored volume is equal to or greater than the common line flush volume value. In one example, the infusion pump 130 can be a fluid displacement pump employing a cassette, such as the Plum 360™ infusion pump available from Hospira, Inc. of Lake Forest, Illinois. Those skilled in the art will appreciate that the infusion pump 130 can be any type of pump operable to drive fluid through the common line 140.

In one embodiment, the infusion pump 130 can be operably connected to a medication management unit (MMU) 170 to receive a drug library including the desired common flush volume value from the MMU 170. In one embodiment, the infusion pump 130 can be further operable to increment a first fluid displayed volume by the monitored volume when the second fluid 122 is driven at the first rate along the second flow path 160. The infusion pump 130 can be further operable to increment a second fluid displayed volume by the monitored volume when the monitored volume is equal to or greater than an internal volume of the common line. In one embodiment, the infusion pump 130 can be operable to stop infusing the first fluid 112 before driving the second fluid 122 at the first rate along the second flow path 160. In one embodiment, the infusion pump 130 can be operable to monitor the volume of the first fluid and switch to delivery of the flush volume when the volume of the first fluid is equal to the received Volume to be Infused (VTBI) for the first fluid. In another embodiment, the infusion pump 130 can be operable to monitor the first fluid path and switch to delivery of the flush volume when the infusion pump detects a given threshold of air (as a single bubble, accumulated bubbles, or by percentage volume) in the first fluid path.

The infusion pump 130 can be operable to receive the common line flush volume value for the common line 140 automatically from the drug library stored in a memory locally in the infusion pump system 100 or remotely on a server. In one example, the drug library associates the common flush volume value with a particular therapeutic agent. In another example, the drug library associates the common flush volume value with a particular clinical care area (CCA), such as general care, an intensive care unit (ICU), a neonatal ICU, or the like. In yet another example, the drug library associates the common flush volume value with a particular consumable infusion set, which directly provides the common line volume. The drug library can include upper and lower dosing limits with hard and soft limits for a number of therapeutic agents. In another embodiment, the infusion pump 130 can be operable to receive the common line flush volume value for the common line 140 from a caregiver via an input on a user interface of the infusion pump. In another embodiment, the infusion pump 130 can be operable to receive the second rate at which the second fluid is delivered when the monitor volume is equal to or greater than the common line flush volume value.

The common line 140 as illustrated includes the line between the junction 180 and the terminal fluid delivery end 140B that is generally connectable at the patient 102, and includes any fluid path common to the first flow path 150 and the second flow path 160. Thus, the common line 140 can include flow paths within the infusion pump 130 (including the associated consumable infusion set, when applicable) common to the first flow path 150 and the second flow path 160, and is not limited to tubing external to the infusion pump 130. The common line 140 is any portion of the infusion pump system 100 through which the first fluid 112 or the second fluid 122 can alternately flow when switched. In one embodiment, the common line flush volume value is an internal volume of the common line 140. Those skilled in the art will appreciate that the common line flush volume value can include an associated consumable infusion set volume, extension sets, filters, patient access devices, catheters, and the like, as required for a particular setup of the infusion pump system 100. In another embodiment, the common line flush volume value is an internal volume of the common line 140 plus an adjustment volume. The adjustment volume can be any volume desired as a safety factor to assure that the common line 140 is free of the first fluid 112 before the second fluid 122 is infused at the second rate.

The infusion pump system 100′ of FIG. 1B has a junction 180′ external to the infusion pump 130′. The infusion pump system 100′ includes a first reservoir 110′ containing a first fluid 112′; a second reservoir 120′ containing a second fluid 122′; a junction 180′ in fluid communication with the first reservoir 110′ and the second reservoir 120′; a common line 140′ in fluid communication between the junction 180′ and the terminal fluid delivery end 140B′ that is generally connectable at the patient 102′, and an infusion pump 130′ operable to drive fluid through the common line 140′. The infusion pump 130′ is operable to: infuse the first fluid 112′ at a first rate along a first flow path 150′ including the first reservoir 110′, the junction 180′, and the common line 140′; receive a common line flush volume value for the common line 140′; switch from the first flow path 150′ to a second flow path 160′ including the second reservoir 120′, the junction 180′, and the common line 140′; drive the second fluid 122′ at the first rate along the second flow path 160′; monitor volume of the second fluid 122′ driven at the first rate; and drive the second fluid 122′ at a second rate along the second flow path 160′ when the monitored volume is equal to or greater than the common line flush volume value. In one embodiment, the junction 180′ can include a two-way valve to manually or automatically switch the infusion pump system 100′ between the first flow path 150′ and the second flow path 160′. In one example, the infusion pump 130′ can be a peristaltic pump, such as the pump used in the Sapphire™ infusion system available from Hospira, Inc. of Lake Forest, Illinois. Those skilled in the art will appreciate that the infusion pump 130′ can be any type of pump operable to drive fluid through the common line 140′.

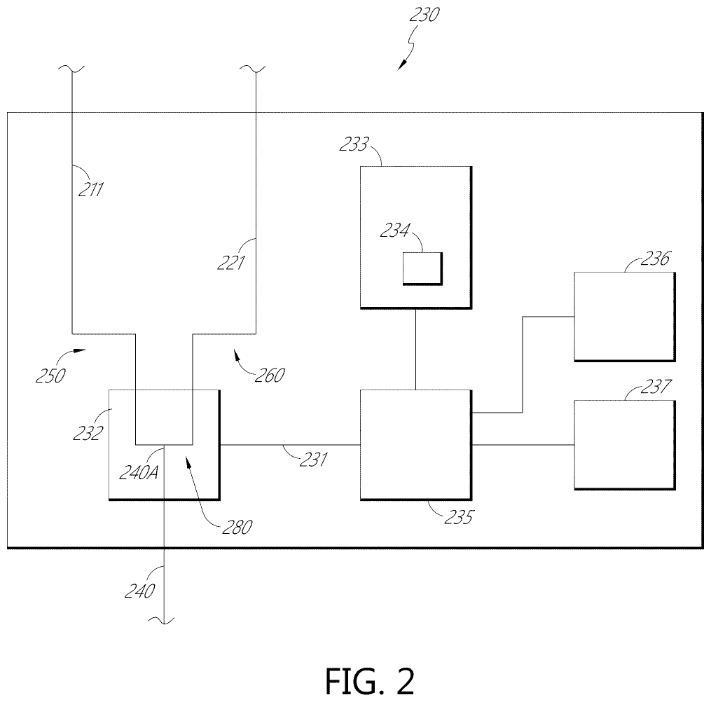

FIG. 2 is a block diagram of an embodiment of an infusion pump with common line auto flush. The infusion pump 230 is operably connected to a common line 240 in fluid communication with a junction 280 at one end 240A and having a terminal fluid delivery end 240B (not shown due to truncation), the junction 280 being in fluid communication with a first reservoir (not shown) containing a first fluid and a second reservoir (not shown) containing a second fluid. In this example, a first reservoir line 211 provides fluid communication between the first reservoir and the junction 280 and a second reservoir line 221 provides fluid communication between the second reservoir and the junction 280.

The infusion pump 230 includes a memory 233 operable to store programming code; a flow controller 235 operably connected to the memory 233; and a fluid driver 232 operably connected to receive a control signal 231 from the flow controller 235, the fluid driver 232 being operable to drive fluid through the common line 240. The flow controller 235 is operable to execute the programming code and provide the control signal 231 to the fluid driver 232 in response to the programming code. The fluid driver 232 is responsive to the control signal 231 to infuse the first fluid at a first rate along a first flow path 211 including the first reservoir, the junction 280, and the common line 240; receive a common line flush volume value for the common line 240; switch from the first flow path 250 to a second flow path 260 including the second reservoir, the junction 280, and the common line 240; drive the second fluid at the first rate along the second flow path 260; monitor volume of the second fluid driven at the first rate; and drive the second fluid at a second rate along the second flow path 260 when the monitored volume is equal to or greater than the common line flush volume value. In an embodiment, the flow controller 235 monitors the volume based on a time elapsed and a rate of delivery. The flow controller 235 can also monitor volume based on measurements, such as number of turns of a motor or signals from a sensor.

The flow controller 235 can be any hardware processor, microprocessor, or the like responsive to the programming code to generate the control signal 231. The fluid driver 232 can be any metered pump, such as a cartridge pump, syringe pump, peristaltic pump, or the like, operable to drive fluid at a desired rate in response to the control signal 231. In one embodiment, the fluid driver 232 can be further responsive to the control signal 231 to increment a first fluid displayed volume by the monitored volume when the second fluid is driven at the first rate along the second flow path 260. The fluid driver 232 can be further responsive to the control signal 231 to increment a second fluid displayed volume by the monitored volume when the monitored volume is equal to or greater than an internal volume of the common line 240. The first fluid displayed volume and/or the second fluid displayed volume can be displayed on a user interface 236. In another embodiment, the fluid driver 232 can be responsive to the control signal 231 to stop infusing the first fluid before driving the second fluid at the first rate along the second flow path 260.

The memory 233 can also be operable to store data, such as a drug library 234 including the common flush volume value, which can optionally be associated with a particular therapeutic agent, a particular clinical care area, and/or a particular consumable infusion set. Different therapeutic agents may have different fluid properties and thus it may be advantageous in some embodiments to associate particular common flush volume value with particular therapeutic agents. In one embodiment, the infusion pump 230 can receive the common line flush volume value for the common line 240 automatically from the drug library 234. In another embodiment, the infusion pump 230 can receive the common line flush volume value manually via direct entry of the value on a user interface 236. The manual entry can be accomplished using a manufacturer provided volume value based upon the length and internal diameter of the common line 240 or a list number or other identifier that is used to access an associated volume value from a lookup table in the pump memory 233, drug library or MMU. The possibility for manual typographical errors can be reduced by use of a barcode, radio frequency (RFID), optical, touch memory reader, near field communicator, or the like to input or scan a machine readable identifier on the infusion set, common line, or its package to obtain the volume value, the list number or other identifier associated with the volume value.

The infusion pump 230 can include human and/or machine interfaces as desired for a particular application. A user interface 236 operably connected to the flow controller 235 can provide input from and/or output to a caregiver or other user to the infusion pump 230. Exemplary user interfaces can include display screens, soft keys or fixed keys, touchscreen displays, and the like. An I/O interface 237 operably connected to the flow controller 235 can provide input from and/or output to hardware associated with the infusion pump 230. Exemplary I/O interfaces can include a wired and/or wireless interface to an electronic network, medication management unit (MMU), medication management system (MMS), or the like.

The common line flush volume value can be selected as desired for a particular application. The common line 240 includes the line between the junction 280 and the terminal fluid delivery end 240B, and includes any fluid path common to the first flow path 250 and the second flow path 260 and so can include any portion of the infusion pump 230 (including the associated consumable infusion set, when applicable) through which the first fluid or the second fluid can alternately flow. In one embodiment, the common line flush volume value is equal to the internal volume of the common line 240, so that the second fluid is infused at the second rate along the second flow path as soon as the first fluid has been cleared from the common line 240. In another embodiment, the common line flush volume value is equal to the internal volume of the common line 240 plus an adjustment volume (to take into account the added/subtracted volume of other connectors or components), so that the second fluid is infused at the second rate along the second flow path after the first fluid has been cleared from the common line 240 plus the adjustment volume of the second fluid has been delivered at the first rate. In another embodiment, the common line flush volume value is equal to the internal volume of the common line modified by a percentage, which could provide a desired overage or underage. The adjustment volume can be used as a safety factor to assure that the common line 240 is free of the first fluid before the second fluid is infused at the second rate.

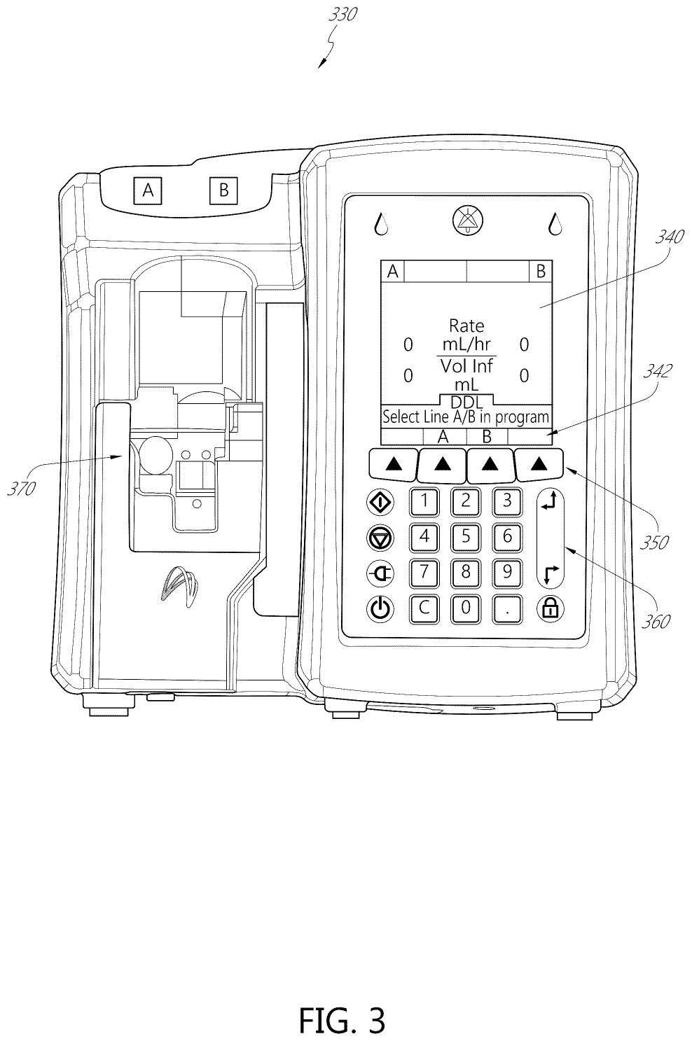

FIG. 3 is a schematic diagram of an infusion pump with common line auto flush in accordance with the present invention. In this example, the infusion pump 330 includes a display 340, soft keys 350, and fixed keys 360 as a user interface. The display 340 provides operational and/or programming information to the user. The soft keys 350 perform different functions depending on the command displayed on an adjacent command portion 342 of the display 340. The fixed keys 360 are labeled with an input or function which functions the same, regardless of whatever is displayed on the display 340. In this example, the infusion pump 330 also includes a pump mechanism 370 operable to communicate with the first reservoir line and the second reservoir line and to move the first fluid or the second fluid to the terminal fluid delivery end of the common line.

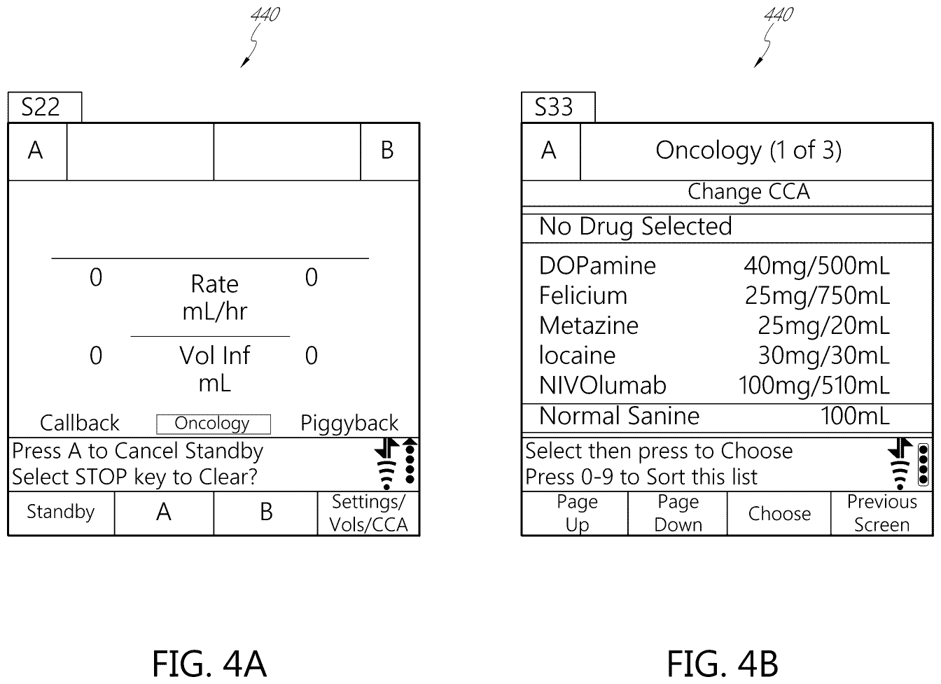

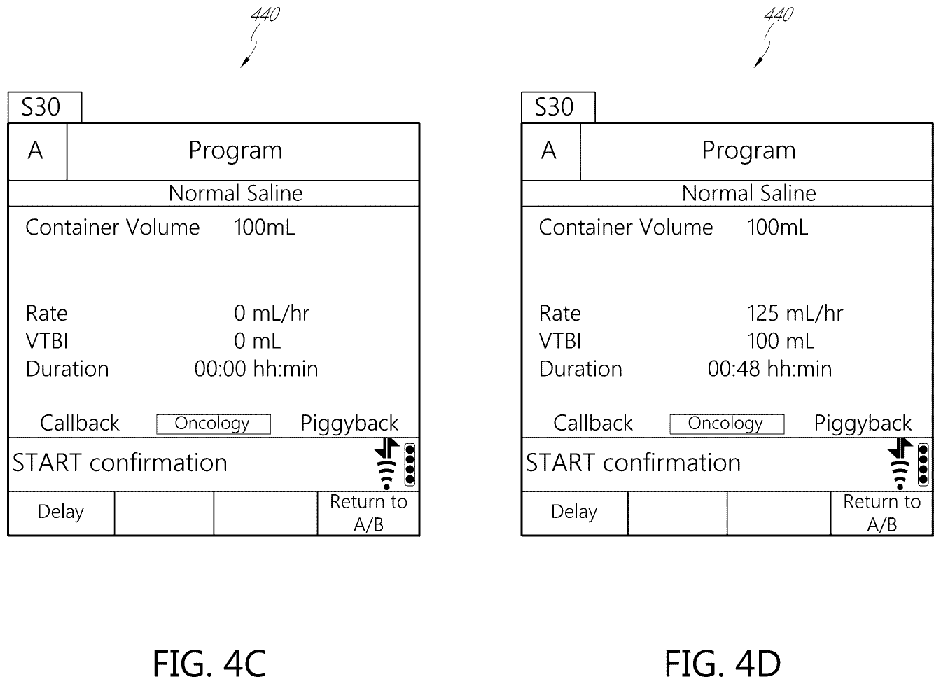

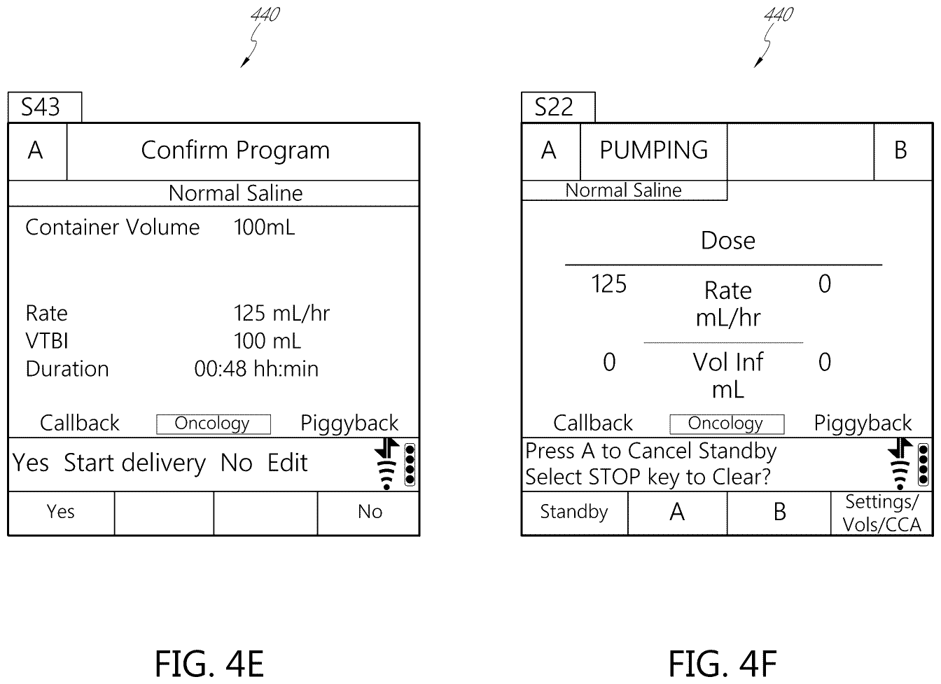

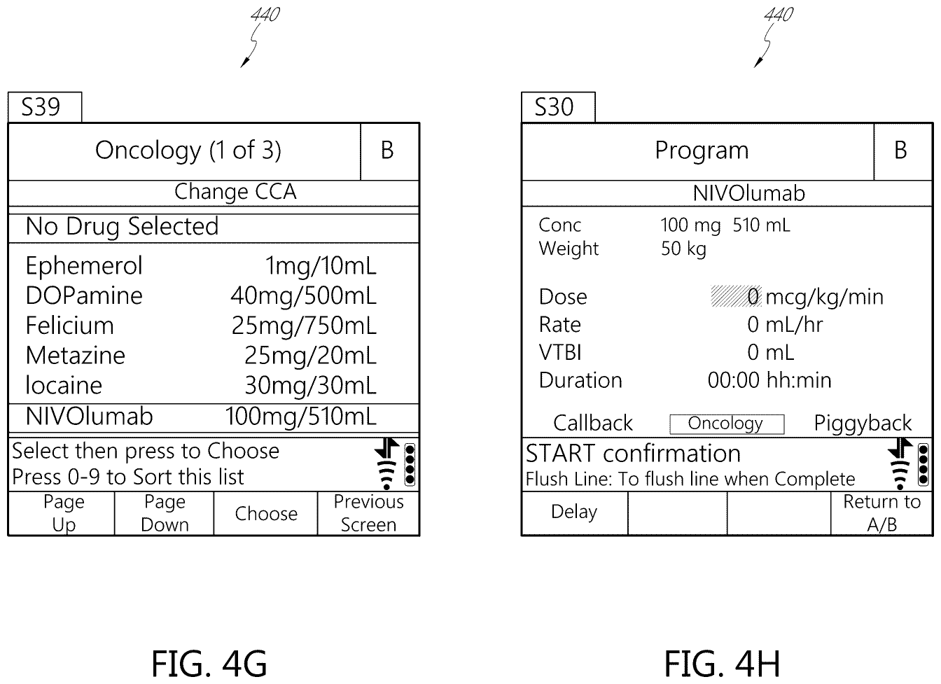

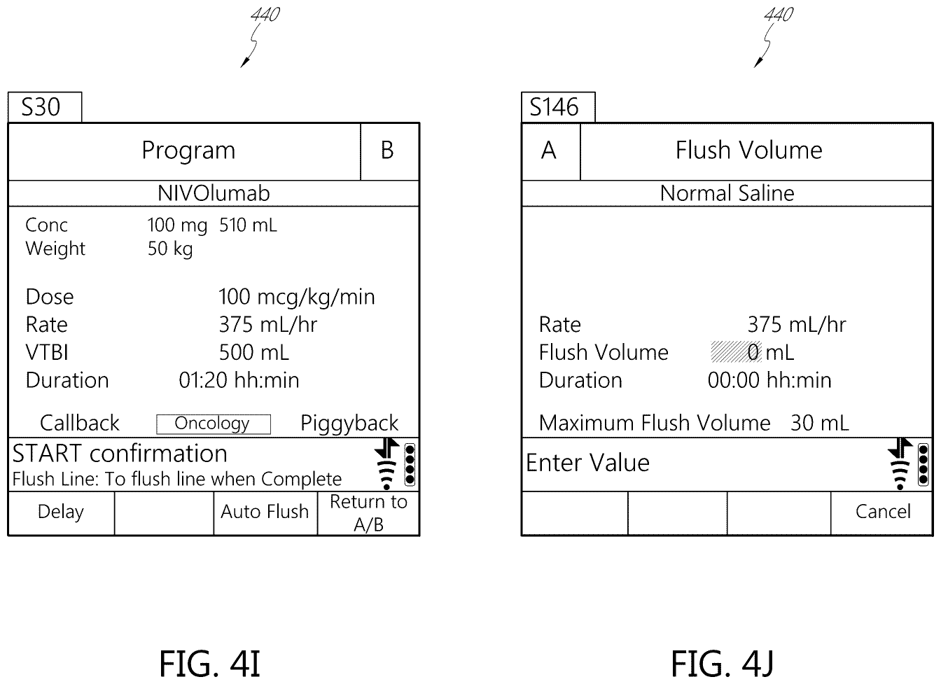

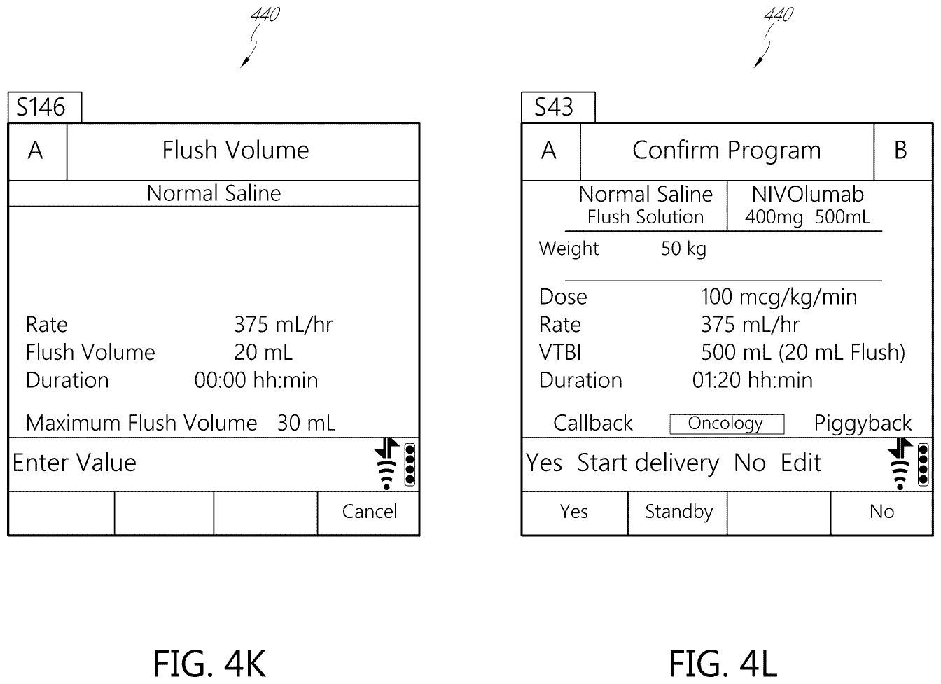

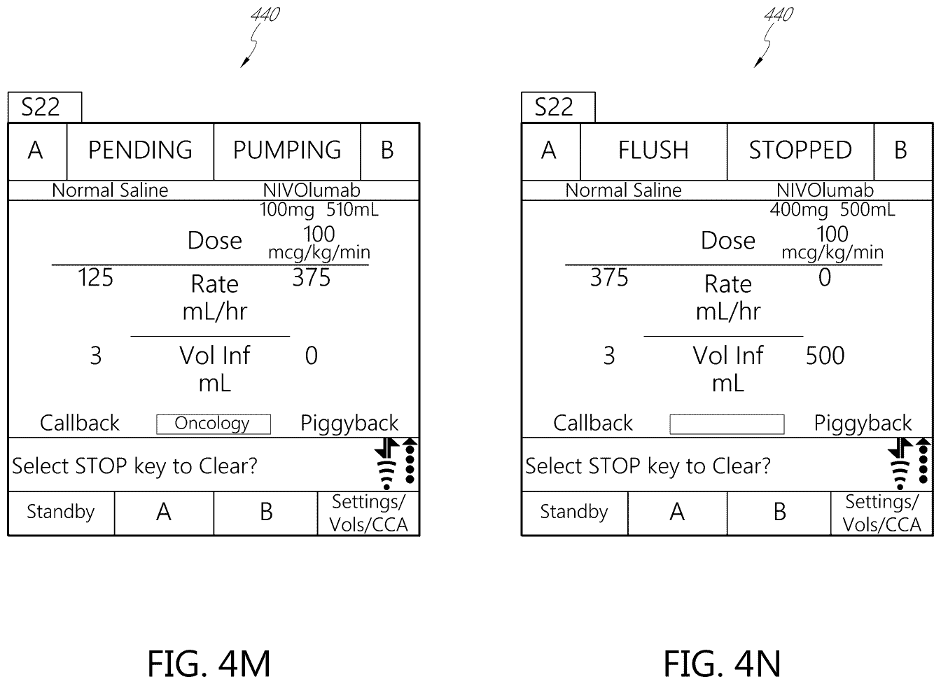

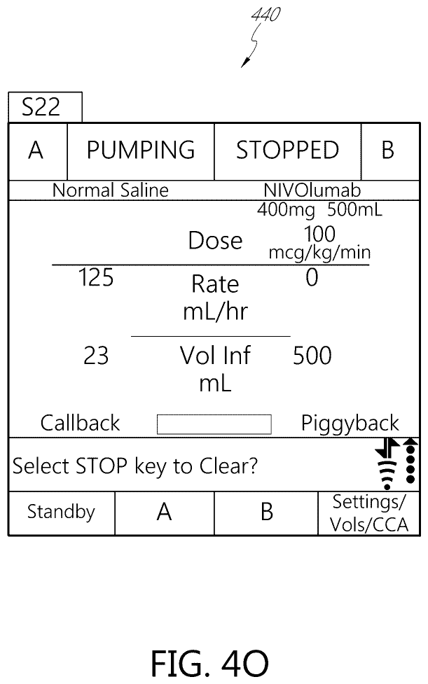

FIGS. 4A-4O illustrate schematic diagrams of user interfaces or screens for an infusion pump system with common line auto flush. In this embodiment, the user can manually edit the common line flush volume value, i.e., the flush volume. In this example, the infusion pump is infusing a first fluid to the terminal fluid delivery end of the common line on Channel A at a first rate, switches to infusing a second fluid to the terminal fluid delivery end of the common line on Channel B at a second rate, then switches back to infusing the first fluid on Channel A but maintains the second rate long enough to clear the remaining second fluid from the common line before changing to the first rate. The flow controller can control the switching using one or more control signals. In some embodiments, the reservoirs may be arranged to switch automatically based on fluid dynamics and arrangement of the reservoirs with respect to each other.

Referring to FIG. 4A, the screen 440 indicates that the infusion pump is in Standby, awaiting programming. The user actuates the soft key associated with Channel A at the bottom of the screen 440 to access a new screen to program Channel A parameters starting with FIG. 4B. Referring to FIG. 4B, the user highlights one of the drugs from a displayed drug list (in this example, Normal Saline) and actuates the soft key associated with Choose at the bottom of the screen 440 to access a new screen to program infusion parameters for the highlighted drug. Referring to FIG. 4C, the user enters values for the Rate, Volume to Be Infused (VTBI), and/or Duration parameters for the infusion of the selected drug (in this example, Normal Saline) for Channel A. Those skilled in the art will appreciate that the parameters can be interrelated, such that the infusion pump automatically fills in the values for some of the parameters once values for other parameters have been entered. Referring to FIG. 4D, the program infusion parameters entered for Channel A are displayed, with a Rate of 125 mL/hr, VTBI of 100 mL and Duration of 00:48 hh:mm. The user actuates the START fixed key to confirm the values entered and to access the Confirm Program screen of FIG. 4E. To start delivery on Channel A, the user actuates the soft key associated with Yes at the bottom of the screen 440 of FIG. 4E, which switches the screen 440 to FIG. 4F indicating that Channel A is infusing the Channel A drug (in this example, Normal Saline) at the Channel A Rate of 125 mL/hr with a Volume Infused of 0 mL.

Referring to FIG. 4F, the user actuates the soft key associated with Channel B at the bottom of the screen 440 to access a new screen to program Channel B parameters starting with FIG. 4G. In this embodiment, the pumping of the first fluid on Channel A continues until the pumping of the second fluid on Channel B is initiated. In another embodiment, the pumping of the first fluid on Channel A can be stopped before pumping the second fluid on Channel B, e.g., while the common line auto flush is being programmed.

Referring to FIG. 4G, the user highlights one of the drugs from a displayed drug list (in this example, NIVOlumab) and actuates the soft key associated with Choose at the bottom of the screen 440 to access a new screen 440 on FIG. 4H to program infusion parameters for the highlighted drug. The user enters values for the Dose, Rate, Volume to Be Infused (VTBI), and/or Duration parameters for the infusion of the selected drug (in this example, NIVOlumab) for Channel B. Those skilled in the art will appreciate that the parameters can be interrelated, such that the infusion pump automatically fills in the values for some of the parameters once values for other parameters have been entered. Referring to FIG. 4I, the program infusion parameters entered for Channel B are displayed, with a Dose of 100 mcg/kg/min, a Rate of 375 mL/hr, a VTBI of 500 mL, and a Duration of 1:20 hh:mm.

To proceed from the screen 440 of FIG. 4I to FIG. 4J to program the common line auto flush parameters, the user actuates the soft key associated with Flush Line (in this example, Auto Flush) at the bottom of the screen 440. In one embodiment, the Flush Line only appears at the bottom of the screen 440 when a first and second fluid are to be infused sequentially through a common line, i.e. for a piggyback infusion and another prior or subsequent infusion. Accordingly, in an embodiment, the flow controller 235 can detect a piggyback infusion and automatically generate a user interface as illustrated to provide an option to select flush parameters. The flow controller 235 may also automatically select flush parameters, such as the common line flush volume from the drug library based on the detection of instructions to switch infusion from the first fluid to the second fluid.

Referring to FIG. 4J, the user can enter the Flush Volume. In this example, the Flush Volume is limited to a maximum flush volume value, such as 30 mL. Those skilled in the art will appreciate that the maximum flush volume value can be selected as desired for a particular therapeutic agent, particular clinical care area, or the like and can be provided through a drug library as desired. Referring to FIG. 4K, the user has entered a value of 20 mL for the Flush Volume. The Rate remains at the previously entered Channel B Rate of 375 mL/hr as shown on FIG. 4I. In this example, the Rate is not editable and the Duration is calculated from the Rate and the Flush Volume. Referring to FIG. 4K, the user actuates the START fixed key to confirm the values entered and to access the Confirm Program screen of FIG. 4L, which shows the Channel B parameters.

In this embodiment, the Flush Volume can be edited by the user. In one example, the initial editable Flush Volume is displayed as a zero value as shown in FIG. 4J. In another example, the initial editable Flush Volume is displayed as a predetermined value, e.g., as a predetermined value provided through a drug library. In another embodiment, the Flush Volume is predetermined and cannot be edited by the user. The Flush Volume can be displayed as in FIG. 4K, but cannot be changed.

Referring to FIG. 4L, the user actuates the soft key associated with Yes at the bottom of the screen 440 to switch from the present Channel A infusion to the Channel B infusion. When the channel is switched, the Channel A drug (in this example, Normal Saline) remaining in the common volume is infused to the terminal fluid delivery end of the common line at the Channel B Rate of 375 mL/hr until the Channel A drug is cleared from the common volume and the Channel B drug (in this example, NIVOlumab) is infused at the Channel B Rate of 375 mL/hr. Referring to FIG. 4M, the Channel A infusion is on hold and Pending and Channel B is infusing the Channel B drug (in this example, NIVOlumab). In this example, the Volume Infused of Channel A drug (in this example, Normal Saline) of 3 mL was infused while the Channel B and flush volume parameters were being set.

Referring to FIG. 4N, Channel B has infused the desired VTBI of 500 mL so Channel B has stopped and Channel A has begun the common line auto flush, continuing the Channel B Rate of 375 mL/hr. When the infusion channel is switched (on reaching the desired Channel B VTBI), the Channel B drug (in this example, NIVOlumab) remaining in the common volume or common line is infused at the Channel B Rate of 375 mL/hr until the Channel B drug is cleared from the common volume or common line. Referring to FIG. 4O, Channel A has delivered the Flush Volume of 20 mL to the patient, clearing the Channel B drug remaining in the common volume or common line. The Channel A drug (in this example, Normal Saline) is then infused at the Channel A Rate of 125 mL/hr. The Volume Infused of Channel A drug (in this example, Normal Saline) has been increased by the Flush Volume of 20 mL (from 3 to 23 mL) to account for the Channel A drug remaining in the common volume or common line previously when switching from Channel A to Channel B.

In this example, the indicated Channel A Volume Infused is lower than the actual Channel A Volume Infused and the indicated Channel B Volume Infused is higher than the actual Channel A Volume Infused from the time of switching the infusion from Channel A to Channel B until the Flush Volume is added to the Channel A Volume Infused following the common line auto flush, the difference between indicated the and the actual being the Flush Volume. The difference is typically small relative to the indicated Volume infused, but those skilled in the art will appreciate that the indicated Volume Infused can be corrected as desired for a particular application.

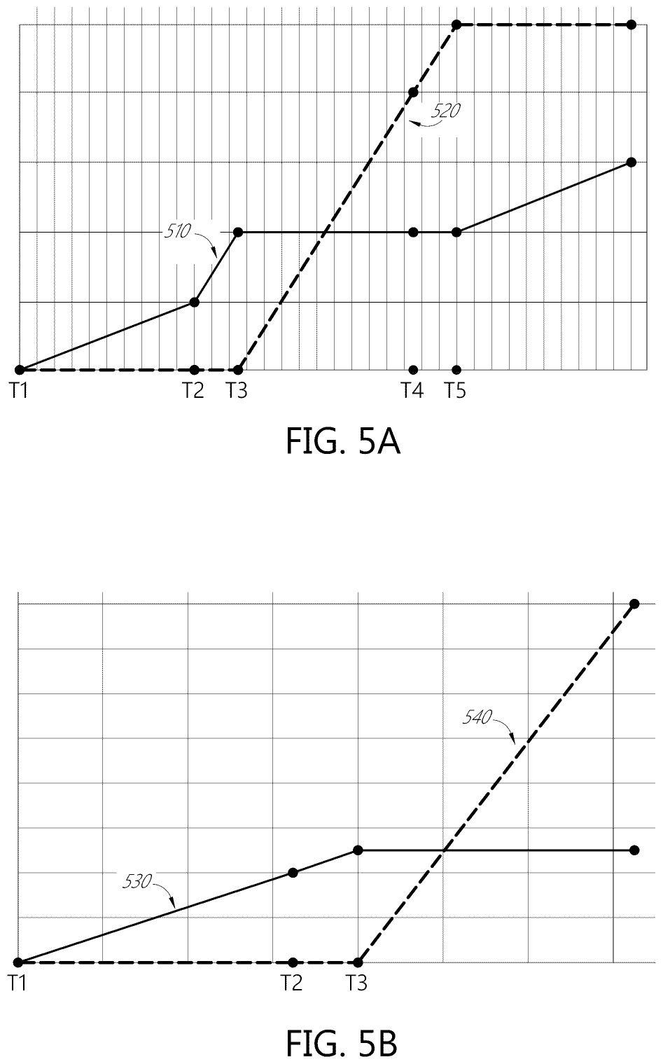

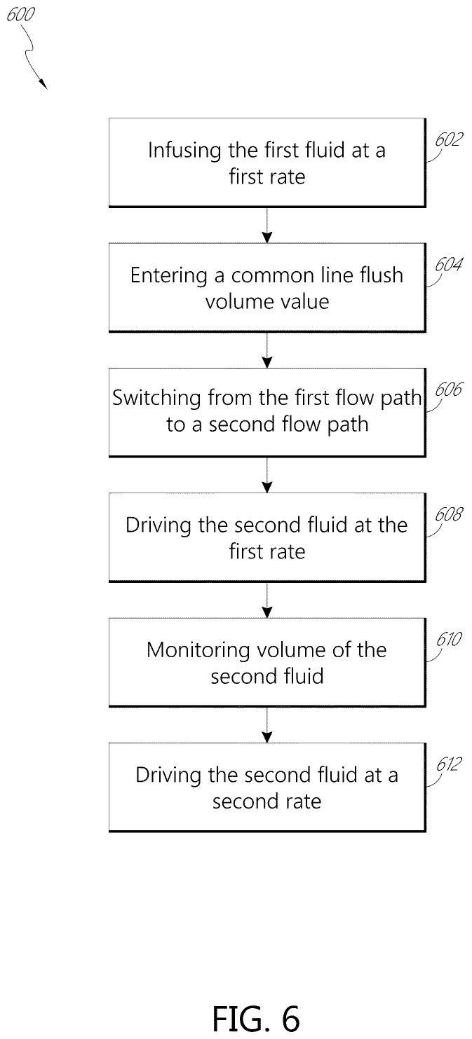

FIGS. 5A & 5B are graphs of fluid volume delivered at the terminal end of the common line or patient versus time for a method of use for an infusion pump with common line auto flush in accordance with the present invention. FIG. 5A illustrates the common line auto flush as described for FIGS. 4A-4O. FIG. 5B illustrates the common line auto flush as described for FIG. 6.

Referring to FIG. 5A, graph 510 is the fluid volume delivered at the terminal fluid delivery end of the common line for a first fluid versus time and graph 520 is the fluid volume delivered at the terminal fluid delivery end of the common line for a second fluid versus time. From T1 to T2, the first fluid is being infused at a first rate along a first flow path including the first reservoir and the second fluid is not being infused. From T2 to T3, the first fluid is being infused at a second rate along a second flow path including the second reservoir as the internal volume of the common line is cleared. The second fluid cannot be infused until the internal volume is cleared of the first fluid. From T3 to T4, the internal volume has been cleared of the first fluid so that no more first fluid is infused and the second fluid is infused at the second rate along the second flow path including the second reservoir. From T4 to T5, the auto flush is performed: the second fluid is infused at the second rate along the first flow path including the first reservoir as the internal volume of the common line is cleared. The first fluid cannot be infused until the internal volume is cleared of the second fluid. After T5, the first fluid is infused at the first rate along the first flow path including the first reservoir after the internal volume of the common line has been cleared of the second fluid. In this example, no additional second fluid is infused after T5.

Those skilled in the art will appreciate that the transition between the two fluids can be selected as desired for a particular application. In the example of FIG. 5A, a common line auto flush is performed from T4 to T5, but not from T2 to T3. As long as the common line flush volume value is known, the common line auto flush maintaining the first rate between T2 and T3 can be performed as desired.

Referring to FIG. 5B, graph 530 is the fluid volume delivered at the terminal fluid delivery end of the common line for a first fluid versus time and graph 540 is the fluid volume delivered at the terminal fluid delivery end of the common line for a second fluid versus time. From T1 to T2, the first fluid is infused at the first rate along a first flow path including the first reservoir and the second fluid is not being infused. From T2 to T3, the flow path is switched from the first flow path including the first reservoir to the second flow path including the second reservoir, and the second fluid is driven at a first rate along the second flow path to perform the common line auto flush. The first fluid is infused, driven or displaced until the internal volume of the common line has been cleared of the first fluid. After T3, the second fluid is infused, driven or displaced at the second rate along the second flow path including the second reservoir after the internal volume of the common line has been cleared of the first fluid. In one embodiment, the common line has been cleared of the second fluid when the monitored volume of the second fluid driven at the first rate between T2 and T3 is equal to or greater than the common line flush volume value. In this example, no additional second fluid is infused after T3.

FIG. 6 is a flowchart of an embodiment of a method for common line auto flush. The method 600 to infuse with an infusion pump system can use an infusion pump system having a first reservoir containing a first fluid, a second reservoir containing a second fluid, a junction in fluid communication with the first reservoir and the second reservoir, a common line in fluid communication with the junction at one end and having a terminal fluid delivery end, and an infusion pump operable to drive fluid through the common line. The method 600 can be performed by any of the systems discussed above. In an embodiment, some or all aspects of the method 600 are stored as programmed instructions to be executed by the flow controller 235. The method 600 can be used with an infusion pump system and infusion pump as described in FIGS. 1A, 1B, & 2 above. In this example, the infusion pump is infusing a first fluid on a first flow path at a first rate and switches to infusing a second fluid on second flow path, maintaining the first rate long enough to clear the remaining first fluid from the common line before changing to a second rate for infusing the second fluid.

Referring to FIG. 6, at block 602, the infusion of the first fluid can be begin at a first rate along a first flow path including the first reservoir, the junction, and the common line. The infusion of the first fluid can be controlled by the flow controller 235 based on a control signal to activate the pump or other mechanical system. In some embodiments, the infusion of the first fluid can also be based on a user input or user control of the pump or the mechanical system. At block 604, the flow controller 235 can receive a common line flush volume value. As discussed above, the common line flush value can be received based on a user input via the user interfaces discussed above. In an embodiment, the flow controller 235 can automatically retrieve the common line flush volume value from the memory 233 or over a network. The common line flush volume may be predetermined for particular fluids. The common line flush volume may also depend on the VTBI or rate of the infusion.

At block 606, the flow controller 235 can determine to switch infusion from the first reservoir to the second reservoir. As discussed above, the infusion may also be switched based on the function of fluid dynamics and arrangement of the respective reservoirs without any determination from the flow controller 235. Switching changes the flow path from the first flow path to a second flow path, which includes the second reservoir, the junction, and the common line. In an embodiment, the flow controller 235 can control a valve to switch the flow path. At block 606, the second fluid is infused at the first rate 608 along the second flow path. The flow controller 235 can use control signals for the infusion of the second fluid and controlling the rate of delivery. By driving the second fluid at the first rate, the first fluid remaining in the common line is flushed and delivered to the patient at the same rate as therapeutically required. In some embodiments, the flow controller 235 obviates the need to specifically arrange the reservoirs by caregivers as the flow controller 235 can control the delivery instead of relying on fluid dynamics and gravity.

At block 610, the flow controller 235 can monitor volume of the second fluid 610 driven at the first rate. The flow controller 235 can determine that the monitored volume is equal to the common line flush volume value. When it's determined that common line flush volume value has been delivered, the flow controller 235 can begin infusion of the second fluid at a second rate along the second flow path as shown in block 612. In some embodiments, the flow controller 235 can track an amount of time before changing the rate of the second fluid delivery to the second rate. In one embodiment, the flow controller 235 can further include incrementing a second fluid displayed volume by the monitored volume when the monitored volume is equal to or greater than an internal volume of the common line. The flow controller 235 can thus accurately track the rate, time, and an amount of fluid delivered to the patient. In some embodiments, the flow controller 235 executes only some of the steps described above with respect to FIG. 6. Furthermore, the flow controller 235 can change the order of the steps, include additional steps, or modify some of the steps discussed above.

The common line flush volume value can be selected as desired for a particular application. In one embodiment, the common line flush volume value is an internal volume of the common line. In another embodiment, the common line flush volume value is an internal volume of the common line plus or minus an adjustment volume. The adjustment volume can be any volume desired as a safety factor to assure that the common line is free of the first fluid before the second fluid is infused at the second rate.

In one embodiment, the method 600 further includes incrementing a first fluid displayed volume by the monitored volume during the infusing the second fluid at the first rate along a second flow path, which can further include incrementing a second fluid displayed volume by the monitored volume when the monitored volume is equal to or greater than an internal volume of the common line.

Entering a common line flush volume value 604 for the common line can be performed manually or automatically. In one embodiment, the entering 604 includes manually entering the common line flush volume value on a user interface of the infusion pump. In another embodiment, the entering 604 includes automatically entering the desired common flush volume value from a drug library. The drug library can associate the desired common flush volume value with a particular therapeutic agent, a particular clinical care area, and/or a particular consumable infusion set.

In one embodiment, the switching from the first flow path to a second flow path 606 including the second reservoir, the junction, and the common line further includes stopping the infusing the first fluid before the driving the second fluid at the first rate along the second flow path.

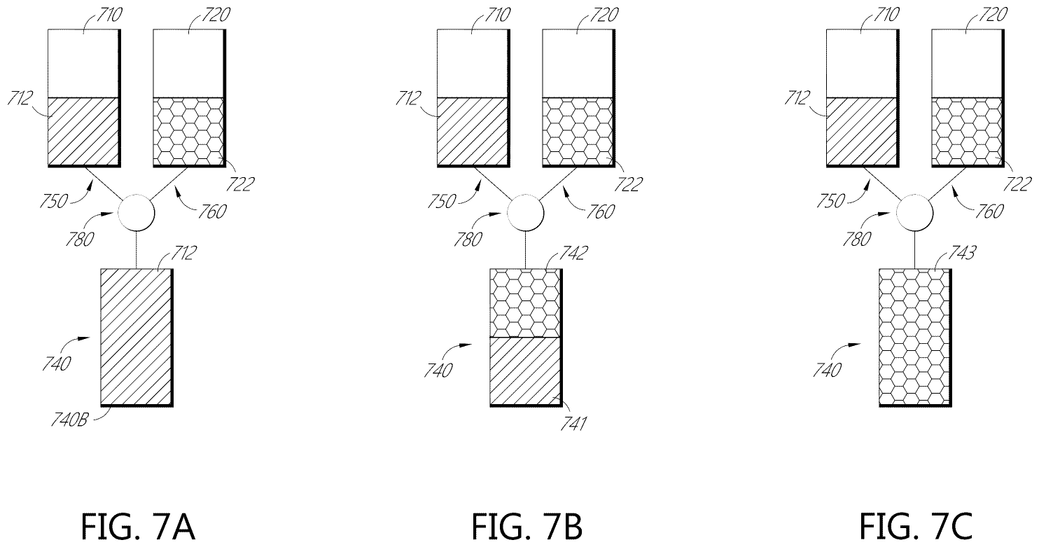

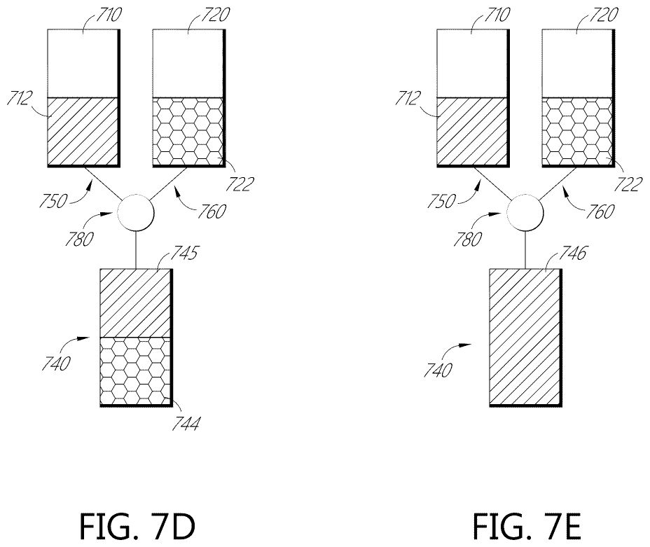

FIGS. 7A-7E are schematic diagrams of use for an infusion pump system with common line auto flush in accordance with the present invention. FIGS. 7A-7E illustrate switching from infusing a first fluid to infusing a second fluid, then switching back to infusing the first fluid, while accounting for the previously infused fluid in the common line. In this example, the infusion pump is infusing a first fluid on a first flow path at a first rate and switches to infusing a second fluid on a second flow path, maintaining the first rate long enough to clear the remaining first fluid from the common line before changing to a second rate for infusing the second fluid. The infusion pump then switches to infusing a first fluid on the first flow path, maintaining the second rate long enough to clear the remaining second fluid from the common line before changing to a first rate for infusing the first fluid.

Referring to FIG. 7A, the first fluid 712 is being delivered to the terminal end 740B at a first rate along a first flow path 750 including the first reservoir 710, the junction 780, and the common line 740. The first fluid 712 is indicated by the diagonal lines. Referring to FIG. 7B, flow has been switched from the first flow path 750 to the second flow path 760 including the second reservoir 720, the junction 780, and the common line 740. The common line 740 contains first common line fluid 741 remaining from the initial infusion and indicated by the diagonal lines, and second common line fluid 742 indicated by the circles. The flow rate remains at the first rate because the remaining first common line fluid 741 is being delivered to the terminal fluid delivery end 740B or to the patient when connected. Referring to FIG. 7C, none of the first fluid remains in the common line 740, so the second common line fluid 743 is driven at a second rate along the second flow path 760.

The use of the infusion pump system can also include switching back to infusing the first fluid. Referring to FIG. 7D, flow has been switched from the second flow path 760 to the first flow path 750. The common line 740 contains second common line fluid 744 remaining from the previous infusion indicated by the circles, and first common line fluid 745 indicated by the diagonal lines. The flow rate remains at the second rate because the remaining second common line fluid 744 is being delivered. Referring to FIG. 7E, none of the second fluid remains in the common line 740, so the first common line fluid 746 is driven at a first rate along the first flow path 750.

While the embodiments of the invention disclosed herein are presently considered to be preferred, various changes, rearrangement of steps, and modifications can be made without departing from the scope of the invention. The scope of the invention is indicated in the appended claims, and all changes that come within the meaning and range of equivalents are intended to be embraced therein.

What is claimed is:

1. A control system for controlling operation of an infusion pump system, the infusion pump system comprising a reservoir configured to hold a first fluid, a fluid line in fluid communication with the reservoir and having a terminal fluid delivery end, and an infusion pump operable to drive fluid through the fluid line toward the terminal fluid delivery end, the control system comprising one or more hardware processors configured to:
receive an instruction to flush the fluid line at a flush rate, wherein the fluid line comprises a fluid line volume of a second fluid;
receive an instruction to infuse the first fluid at an infusion rate from the reservoir to the terminal fluid delivery end;
flush the fluid line by infusing a fluid line volume of the first fluid from the reservoir at a flush rate, wherein the flush rate is greater than the infusion rate;
determine that the fluid line volume of the first fluid has been infused at the flush rate, wherein the fluid line is substantially free of the second fluid; and
in response to determining that the fluid line volume of the first fluid has been infused at the flush rate, change to infusing the first fluid from the reservoir at the infusion rate.

2. The control system of claim 1, wherein the fluid line volume is received as part of the instruction to flush the fluid line.

3. The control system of claim 1, wherein the fluid line volume is retrieved from a memory in response to receiving the instruction to flush the fluid line.

4. The control system of claim 1, wherein the fluid line volume is retrieved over a network.

5. The control system of claim 1, wherein the fluid line volume is predetermined.

6. The control system of claim 1, wherein the fluid line volume is based on the first fluid.

7. The control system of claim 1, wherein the flush rate is at least three times as great as the infusion rate.

8. The control system of claim 1, wherein the instruction to flush the fluid line and the instruction to infuse the first fluid are received from an input via a user interface.

9. The control system of claim 1, wherein the infusion pump system comprises a second reservoir configured to hold the second fluid, and a junction in fluid communication with the reservoir and the second reservoir, and wherein the fluid line is in fluid communication with the junction.

10. The control system of claim 9, wherein the one or more hardware processors are further configured to control a valve, wherein the valve is configured to switch from infusion of the second fluid along a second flow path from the second reservoir to infusion of the first fluid along a first flow path from the reservoir.

11. A method for controlling operation of an infusion pump system, the infusion pump system comprising a reservoir configured to hold a first fluid, a fluid line in fluid communication with the reservoir and having a terminal fluid delivery end, and an infusion pump operable to drive fluid through the fluid line toward the terminal fluid delivery end, the method comprising:
receiving an instruction to flush the fluid line at a flush rate, wherein the fluid line comprises a fluid line volume of a second fluid;
receiving an instruction to infuse the first fluid at an infusion rate from the reservoir to the terminal fluid delivery end;
flushing the fluid line by infusing a fluid line volume of the first fluid from the reservoir at a flush rate, wherein the flush rate is greater than the infusion rate;
determining that the fluid line volume of the first fluid has been infused at the flush rate, wherein the fluid line is substantially free of the second fluid; and
in response to determining that the fluid line volume of the first fluid has been infused at the flush rate, changing to infusing the first fluid through from the reservoir at the infusion rate.

12. The method of claim 11, further comprising obtaining the fluid line volume from the instruction to flush the fluid line.

13. The method of claim 11, further comprising obtaining the fluid line volume from a computer-readable memory.

14. The method of claim 11, further comprising obtaining the fluid line volume over a network.

15. The method of claim 11, wherein the fluid line volume is predetermined.

16. The method of claim 11, wherein the fluid line volume is based on the first fluid.

17. The method of claim 11, wherein the flush rate is at least three times as great as the infusion rate.

18. The method of claim 11, wherein the instruction to flush the fluid line and the instruction to infuse the first fluid are received from an input via a user interface.

19. The method of claim 11, wherein the infusion pump system comprises a second reservoir configured to hold the second fluid, and a junction in fluid communication with the reservoir and the second reservoir, and wherein the fluid line is in fluid communication with the junction.

20. The method of claim 19, further comprising controlling a valve to switch from infusion of the second fluid along a second flow path from the second reservoir to infusion of the first fluid along a first fluid path from the reservoir.
